(19) United States
(12) Patent Application Publication
(10) Pub. No.: US 2008/0108866 A1
(43) Pub. Date: May 8, 2008

(54) Control method for capsule endoscope with memory storage device

(76) Inventor: Feng-Chuan Lin, Taipei (TW)

Correspondence Address: BIRCH STEWART KOLASCH & BIRCH, PO BOX 747, FALLS CHURCH, VA, 22040-0747, US

(21) Appl. No.: 11/593,112

(22) Filed: Nov. 6, 2006

(51) Int. Cl. A61B 1/00

(52) U.S. Cl. 600/103

(57) ABSTRACT

The object of the present invention is to reveal how to control the operation of a capsule endoscope that has a memory storage device. The capsule endoscope is swallowed through the mouth to start the photographic inspection. During the process of operating the capsule endoscope, the built-in wireless receiving module is used to receive instructions and further to adjust the movement of capsule endoscope in order to achieve the inspection tasks; on completion of the photographic inspection, the capsule shell is cut open and connected to the host computer, and the image and data stored in the storage module are accessed through the host computer.

### FIELD OF THE INVENTION

The present invention relates to a kind of capsule endoscope, and more particularly to the control method for a kind of capsule endoscope with memory storage device.

### BACKGROUND OF THE INVENTION

Since 1975 many medical scholars have been engaging in the examination of digestive system. Under the situation where the examining equipment is more primitive and inconvenient, usually on the front and back portion of the digestive system can be examined. Subsequently, for the improvement of ease in examination, there is the conception and invention of endoscope. The American company called Welch-Ally invented electronic endoscope in 1984. With advances in the hi-tech industry, there are also significant improvement and progress in endoscope and evolves to the apparatus of endoscope examination today. This refers to the traditional endoscope, meaning a piece of long black tube. One end of the tube is connected to the machine. This machine could produce cold light source. The tube has optical fiber inside and could transmit the cold light to the other end of the tube through optical fiber. During examination, a tube is inserted into the organ to be examined. The cold light transmitted from cold light source could light up the inside of the organ. The optical sensor chip (CCD, similar to DV or V8 video recorder) fixed at the end of the tube could send the clear image signal back to the machine. The computer in the machine will convert the signal into image. In this way, we could see the inside of the organ just as watching TV. The doctor will then control the direction of the tube (traditional endoscope) to the left, right, or moving forward and easily maneuver the area intended to see. However, there is still a large section of the intestine that could not be easily checked with endoscope. This is because if the length of the endoscope is too long, it is harder to maneuver, and patent has to endure more pain. This is clearly not a good method of examination.

With the advances in technology, a technique using biological wireless remote control is gradually being developed and abandoning the prior wired transmission. The size and method of usage are also being resolve to overcome the drawback of the present endoscope. In recent years, with the invention of energy saving and miniaturizing of optical sensor chip, there is the birth of small size capsule endoscope. Such capsule endoscope refers to the size of optical sensor chip for photographic camera and the cold light source are shrunk to the size of capsule pill with the dimension of 11×26 mm. Except for the optical sensor chip inside, there are small battery, LED, and computer chip and radio transmitter. The camera can take two pictures every second. The examination time is about 8 hours each time. A total of 50,000 pictures can be taken. The endoscope, same size of a capsule, is swallowed into the stomach. Inside the body, the capsule advances with the movement of the intestine. Meanwhile, the optical sensor could take pictures at the same time to obtain the image of interior of the organ. The image signals obtained by the optical sensor chip is transmitted to outside of the body through the use of radio apparatus. We will set up 9 pieces of radio antenna on the abdomen of the person receiving examination, and store the received signals in the portable receiver. The capsule endoscope will then faithfully recording all the images it takes from esophagus all the way through small intestine till the large intestine until it runs out of battery. Finally, the endoscope is discharged through the anus with stool and concludes the whole examination mission. In this way, it is very convenient to examine a patient.

However, the cost involved in the installation of capsule endoscope and its peripheral devices for wireless transmission is very expensive, and specialist personnel are required to install large size of antenna and receiving storage device on the person being examined in order to receive image information. Such process is quite complicated, and it is quite inconvenient to move around for the person being examined with such large volume of antenna and receiver. This makes it harder to promote the use of such capsule endoscope system.

Besides, the antenna is stuck to the person receiving the examination for the collection of data, which will have the problem of sensitivity of receiving. For example, the one-meter range between intestine and pylorus is less sensitive for the capsule endoscope to transmit radio information.

As to the accuracy aspect, as the frequency required for the radio transmission in human body is in low frequency, it is not possible to use large width of information for the transmission. Thus, the number of pictures transmitted and resolution is greatly restricted. As the information could not be transmitted in large amount, it is not possible to transmit larger number of pictures. For instance, in the area of esophagus where the movement is faster, at most two pictures per second can be transmitted. Some data can be lost if the setup is moving too fast. In addition, such setup cannot be installed with too many photo sensor chips for the capturing of more image data and leaving more data for the doctor to make the judgement. If two (or more) optical sensor can be installed on the front and back sides of the capsule endoscope in the geometrical method, it is possible to obtain about 360 degrees of image on the surrounding of capsule endoscope. Thus it is possible to drastically increase the information of images and increase the accuracy of doctor's interpretation of data.

In view of this, this invention is a kind of control method for capsule endoscope with memory storage device in order to resolve the problems of the traditional technology.

### SUMMARY OF THE INVENTION

A primary object of the present invention is to provide a control method for capsule endoscope with memory storage device; a unidirectional wireless receiving module is added to the internal of the capsule endoscope for receiving external instructions in order to adjust the movement of the capsule endoscope so ask to achieve certain particular sampling task and further to drastically increase the accuracy of examination.

Another object of the present invention is to provide a wireless unit for sending unidirectional instruction to the capsule endoscope, which is a very small size magnetic material that is harmless to human body, or low frequency, low power unidirectional wireless unit for sticking to the person's body that instructions must be sent to the capsule endoscope, on receiving the instruction what the form of action pictures must be taken in order to achieve certain particular sampling tasks.

Still another object of this invention for expanding other testing units inside the capsule endoscope in order to obtain other testing information for the purpose of securing more information to increase doctor's judgement and sending instructions to the capsule endoscope to activate or stop the examination device by using the control method described above.

To achieve the above objects, this invention is to swallow the endoscope the size of a capsule into the stomach. Inside the stomach, the capsule endoscope will advance with the movement of the intestine. The capsule endoscope itself will flash at the rate of two times per second so that the optical sensor chip could take the picture at the same time to obtain the image of interior of organs. The image signals obtained from the optical sensor chip will be written into the memory storage device at very high speed. The capsule endoscope will then record all pictures taken inside the stomach faithfully until the battery runs out of power. Then, it will be discharged through anus with stools and conclude the whole inspection mission. However, before such examination can be carried out, special requirement must be followed and the person's body must be stuck with different “wireless transmitting units”. When the capsule endoscope receives an instruction, it will immediately adjust the movement of capsule endoscope in order to accomplish certain special sampling tasks.

On completion of the examination, the capsule endoscope discharged will be cleaned and sterilized. The shell of the capsule will be cut open and the host interface of this device will be connected to the terminal host. The controller of the capsule endoscope will communicate with the terminal host through the host interface. On turning on of power, the controller will read from the information block of SRAM into the controller. Based on the data downloaded from the information block of the SRAM, the controller will respond based on the request of the host and configure the memory module and regards it as logical disk. The host could access all pictures taken freely through the logical disk.

Below will further explain through the attached diagram of the embodiment in order to understand the object of this invention, description of technology, features, and the performance achieved.

### BRIEF DESCRIPTION OF THE DRAWINGS

FIG. 1 is the illustration of the embodiment of the newly added wireless receiving module capsule endoscope with build-in memory storage device;

FIG. 1A is the 3D diagram of the newly added wireless receiving module of the capsule endoscope with memory storage device of this invention;

FIG. 2 is the illustration of the state of the capsule endoscope of this invention in operational control;

FIG. 3 is the illustration of the state of this invention in connection with a terminal host.

### DETAILED DESCRIPTION OF THE PREFERRED EMBODIMENTS

For the ease in understanding other characteristics of this invention and the advantages as well as to exemplify the achieved performance for the auditing committee. This invention together with the illustration is described in detail as shown below.

Capsule endoscope containing large amount of storage device is using the controller to integrate optical sensor chip (contains focusing lens) and memory module. The method of data bus interface is used to read the image data of optical sensor chip controlled by the controller, then use very high speed to write into the memory module on the same data bus and further to drastically increase the number of pictures processed per second. Here there could be more optical sensor chips combined to take 360 degrees image. In this way, it is possible to increase the information of the image and increase doctor's read-out accuracy.

However, as there is limitation in the size of capsule endoscope and the electrical power stored, a method that could efficiently utilize the limited power and memory of the endoscope is needed in order to achieve maximum benefit and most information to help doctor in reading the data.

On the other hand, capsule endoscope using this new structure could cause the controller to integrate other testing unit (such as acid and alkaline value testing device) in additional to the optical sensor chip (containing focusing lens) and then directly write the newly added testing information from controller to memory module. After the testing is completed and the device is discharge from the body, the terminal host is used to download the pictures taken and the additional information in order to provide rich information for doctor to read-out.

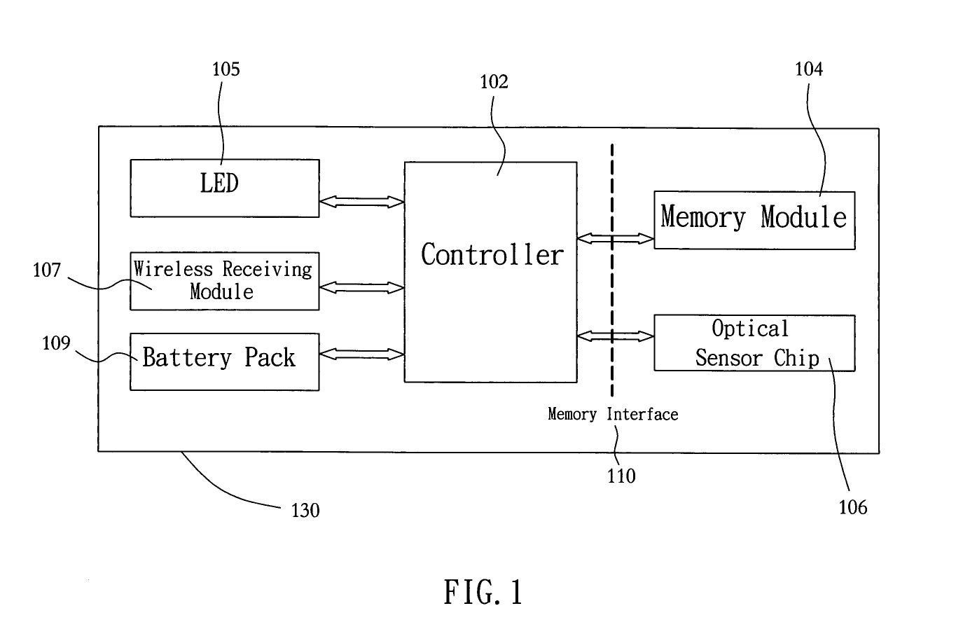

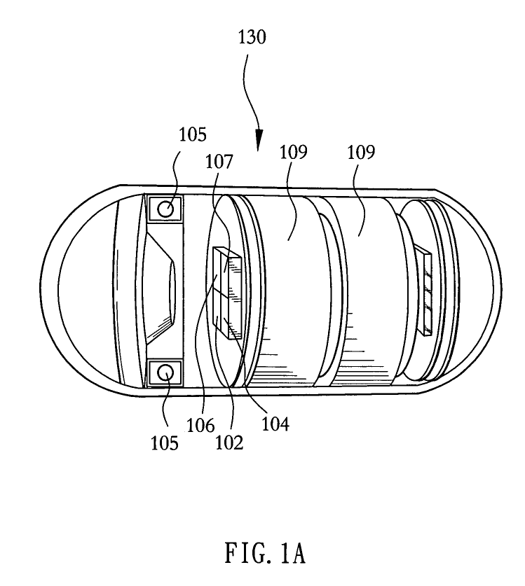

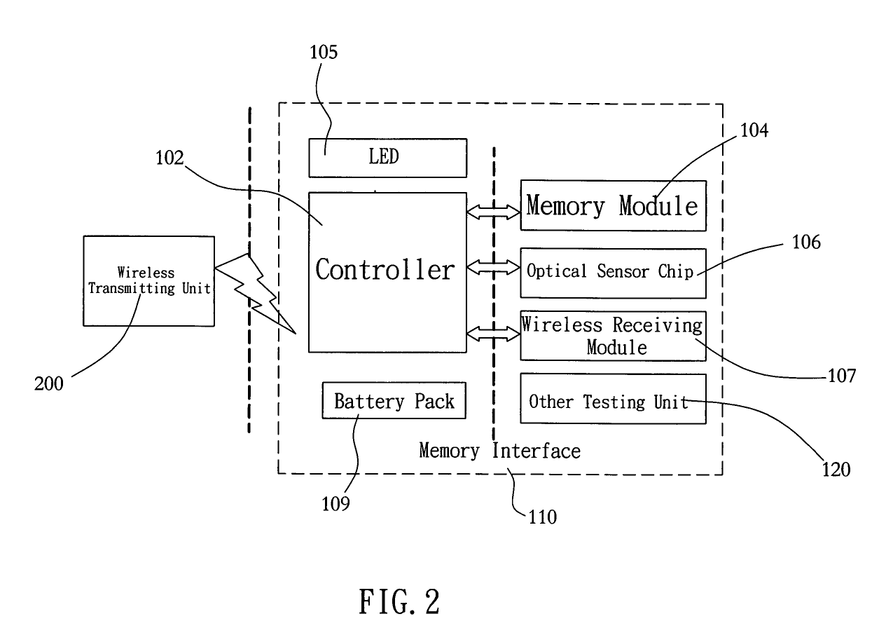

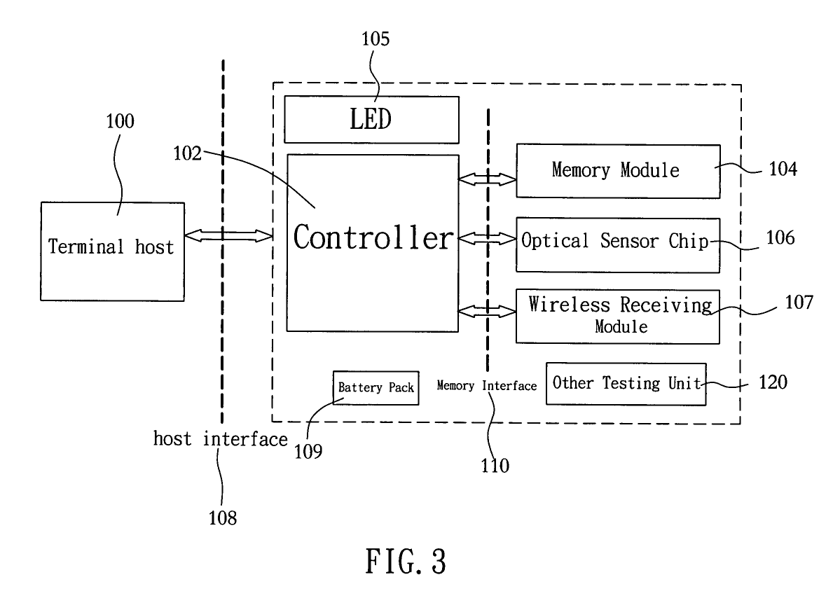

Please refer to FIG. 1 that is the illustration of the embodiment of the present invention of capsule endoscope with newly added wireless receiving module containing memory storage device. FIG. 1A is the 3D illustration of the capsule shell of the capsule endoscope containing memory storage device with newly added wireless receiving module. FIG. 2 is the illustration of the state of the capsule endoscope in the control operation of this invention, and FIG. 3 is the illustration of the connecting state of this invention with a terminal host. As shown in these figures, the capsule endoscope with memory storage device has a memory interface 110 containing a controller 102, a memory module 104, an LED 105, an optical sensor chip 106 (containing focusing lens), a wireless receiving module 107, a battery pack 109 providing power, and the acid-alkaline resisting capsule shell 130 for enclosing the above stated components (picture taking opening is transparent) and a host interface 108.

The task of controller 102 is to communicate with terminal host 100 and at the same time manage memory module 104, LED 105, optical sensor chip 106 (contains focusing lens), and wireless receiving module 107. Memory module 104 must contain at least one storage chip or memory for storing information, such as flash memory, PROM, or any EPROM; when activating the power of the battery pack 109 in the device, the controller 102 will start to drive LED 105 and optical sensor chip 106 (contains focusing lens) and engage in the photographing process and at the same time store the images taken into memory module 104; controller 102 at the same time also receives the state changes or information from wireless receiving module 107 in order to obtain the instructions given externally and then changes the current operating mode of controller 102 so as to meet the operating mode intended externally; if the esophagus is 90 degrees vertical, the image of esophagus almost unable to obtain effectively. If this device could activate two cameras since the beginning to take images of almost 360 degrees and at the same time the number of pictures taken is increased to more than 10 pieces, then all images in the esophagus will all be taken without miss even if moving in high speed in the esophagus; and we stick a “recovering” wireless transmitting unit 200 (not illustrated in the figure) at the connection between esophagus and stomach initially. On receiving instruction from capsule endoscope, it will immediately adjust the action of capsule endoscope immediately and recover back to taking two pictures each second. In this way, it is possible to reduce power consumption, safe storage space, and at the same time satisfy the requirement of certain portion need to secure more information.

Controller 102 will manage memory module 104 at the same time and store the several images taken into one picture file; then the capsule endoscope will faithfully record all pictures taken in the stomach all the way from esophagus to small intestine or even to large intestine until the battery runs out and discharged with stool from anus and conclude the whole examining task.

Then, the discharged capsule endoscope will be cleaned and sterilized, the capsule shell 130 is cut open, and the host interface 108 of the device is connected to the terminal host 100. The controller 102 in the capsule endoscope communicates with the terminal host 100 through the host interface 108. Controller 102 on connecting to terminal host 100 and obtaining the power will read the information block into the SRAM of controller. According to the information block information loaded into SRAM, controller 102 will react to the request issued by the terminal host 100 and configure the memory module, and treat it a logical disk. The host will access all image files freely through the logical disk. The application software in the terminal host 100 will operate in multiplex mode and retrieve all image files of memory module 104, at the same time it will display the retrieved image file on the display instantly, and save or make into CD file after processing the displayed image file.

In addition, the controller 102 can further integrate with some other test unit 120 other than optical sensor chip such as acid or alkaline testing device and then directly write the newly added acid or alkaline testing value into memory by the controller 102. When the testing is completed the device is discharged out of the body and the terminal host 100 is used to download the images taken and the testing information of the added acid and alkaline values so as to provide rich information for doctor's read-out.

To achieve the object of the above stated invention, this invention is to swallow the endoscope the size of a capsule into the stomach. In the stomach, the capsule endoscope will advance with the movement of the intestine, and the capsule endoscope itself will flash twice per second. At this time, the optical sensor chip will take pictures at the same time to obtain the images inside the organ and use very high speed to write the image signals obtained by the optical sensor chip into the memory storage device. Then, the capsule endoscope will faithfully record all pictures taken in the stomach from esophagus all the way to the small intestine or even to the large intestine until finally the battery runs out of power and discharged from anus with stool and conclude the whole examining mission. However, before executing the test, special requirement can be followed and then sticks different “wireless transmitting units” on the body. When the capsule endoscope actually receives instruction, it will adjust the action of the capsule endoscope instantly in order to achieve certain particular sampling task. On completing the test, the capsule endoscope is cleaned and sterilized before the capsule shell is cut open and connected to the terminal host through the host interface. After turning on the initial power, the controller will read the information block into the SRAM of controller. Based on the information block data is loaded into SRAM, the controller will respond to the request from the host and configure the memory module and treat it as the logical disk. The host will access all pictures freely through the logical disk.

In summary, this invention has achieved breakthrough under the prior technical structure and indeed has achieved all intended effect and is not easy for person familiar with the art to think of. In addition, the application of this innovation has never be opened, and the advances, the practicality of this invention obviously has meet the requirement of applying for invention patent. Therefore, the invention is applying based on the law and request your authority to grant the invention patent application for encouraging the innovation. We are appreciative of your approval.

The embodiment stated above is for explaining the thinking and features of this invention. Its purpose is for the people skilled in this art to understand the content of this invention and implement accordingly. It certainly is not limited to the range of the patent of this invention. All equivalent variations or modifications based on the spirit revealed by this invention shall be covered in the patent of this invention.

What is claimed is:

1. A control method of capsule endoscope with memory storage device, including a capsule shell; the interior of the said capsule shell has at least one controller responsible for connecting and control an LED, a wireless receiving module, a battery pack, a memory module, and an optical sensor chip (contains focusing lens); firstly the said battery pack provides power to the said LED to light up external world, then through the said wireless receiving unit to receive the instruction of outside world and transmit to the said controller, activate the said optical sensor chip to engage in the action of taking picture, and lastly to store the information taken into the said memory module; and
An wireless transmitting unit, responsible for transmitting the signal to the said wireless receiving unit inside the said capsule shell, and further to control the said controller in engaging in versatile sampling action.

2. As the control method of capsule endoscope with memory storage device stated in item 1 of the patent application range, where the said controller can be expanded to connect and control one other testing unit for increasing the sampling function of the said capsule endoscope.

3. As the control method of capsule endoscope with memory storage device stated in item 1 of the patent application range, where the said testing unit can be an acid and alkaline value testing device.

4. As the control method of capsule endoscope with memory storage device stated in item 1 of the patent application range, where the said capsule endoscope instantly adjust the number of times of taking pictures of the said optical sensor chip through the signal instructions actually received, thus is able to lower the power consumption, save storage space, at the same time could satisfy the requirement of certain portion needs to secure more information.
